(19) United States
(12) Patent Application Publication
(10) Pub. No.: US 2017/0143720 A1
(43) Pub. Date: May 25, 2017

(54) CALCIUM FOLATE (CAFOLATE) AND THERAPEUTIC METHODS BASED THEREON

(71) Applicant: SanRx Pharmaceuticals, Inc., La Jolla, CA (US)

(72) Inventor: Phillip Moheno, San Diego, CA (US)

(21) Appl. No.: 15/353,527

(22) Filed: Nov. 16, 2016

Related U.S. Application Data
(63) Continuation of application No. 14/375,411, filed on Jul. 29, 2014, now abandoned.
(60) Provisional application No. 61/592,510, filed on Jan. 30, 2012.

(51) Int. Cl. A61K 31/519; A61K 45/06

(52) U.S. Cl. CPC A61K 31/519; A61K 45/06

(57) ABSTRACT

Disclosed herein are methods for the treatment of cancer and inflammatory-based diseases and disorders, such as hepatitis B virus infection, tuberculosis and type 2 diabetes based upon the administration of CaFolate. In one embodiment is a method of treating cancer comprising administration of CaFolate. In another embodiment is a method of treatment inflammatory-based disease and disorders comprising administration of CaFolate.

### CROSS REFERENCE TO RELATED APPLICATIONS

This application claims the benefit of U.S. Application Ser. No. 61/592,510, filed Jan. 30, 2012, which is hereby incorporated by reference in its entirety.

### SUMMARY OF THE INVENTION

This application provides CaFolate and therapeutics methods based thereon.

In one embodiment CaFolate is an immune-modulator via MO or TDO (tryptophan 2,3-dioxygenase) pathways. In another embodiment is a method of treating cancer comprising administration of a composition comprising CaFolate. In another embodiment is the method wherein the CaFolate in-vivo transformed to calcium pterin [CaPterin].

In another embodiment is the method wherein administration of the CaFolate results in decreased IL-6 levels. In another embodiment is the method wherein administration of the CaFolate results in increased IL-10 levels. In another embodiment is the method wherein administration of the CaFolate results in decreased IFN-γ levels. In another embodiment is the method wherein administration of the CaFolate results in increased kynurenine levels. In another embodiment is the method wherein administration of the CaFolate results in increased IL-12 levels. In another embodiment is the method wherein administration of the CaFolate results in decreased IL-6 levels. In another embodiment is the method wherein administration of the CaFolate results in increased IL-4 levels.

In another embodiment is the method wherein administration of the CaFolate results in inhibition of indoleamine 2,3-dioxygenase.

In another embodiment is the method wherein administering the CaFolate is through oral, parenteral, intravenous, subcutaneous, intrathecal, intramuscular, buccal, intranasal, epidural, sublingual, pulmonary, local, rectal, or transdermal administration.

In another embodiment is the method further comprising additional therapies selected from one or more of radiation therapy, chemotherapy, high dose chemotherapy with stem cell transplant, hormone therapy, and monoclonal antibody therapy.

In another embodiment is the method wherein the cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In another embodiment is a method of modulating the immune response comprising administration of a composition comprising CaFolate.

In another embodiment is a method of treating an inflammatory-based disease or disorder comprising administration of a composition comprising CaFolate. In another embodiment is the method wherein the inflammatory-based disease or disorder is selected from infectious diseases, neurodegenerative disorders, multiple sclerosis, HIV-associate dementia, AIDS dementia, Alzheimer's disease, central nervous system inflammation, obesity, dementia (various forms), coronary heart disease, diabetes (Type 1 and Type 2), atherosclerosis, chronic inflammatory diseases, autism, neonatal onset multisystem inflammatory disease, (also known as NOMID, Chronic Neurologic Cutaneous and Articular Syndrome, or CINCA), Parkinson's Disease, rheumatoid arthritis, osteoarthritis, tendinitis, bursitis, inflammatory lung disease, psoriasis, chronic obstructive pulmonary disease, lupus erythematosus, organ inflammation (e.g. myocarditis, asthma, nepHritis, colitis), inflammatory bowel disease (IBD), autoimmune disease, inflammatory bowel syndrome (IBS), Crohn's Disease, Chronic Ulcerative Colitis, transplant rejection, sepsis, disseminated intravascular coagulation (DIC), septic shock, psoriasis, emphysema and ischemia-reperfusion injury. In another embodiment is the method wherein administering the composition is through oral, parenteral, intravenous, subcutaneous, intrathecal, intramuscular, buccal, intranasal, epidural, sublingual, pulmonary, local, rectal, or transdermal administration.

In another embodiment is the method wherein the inflammatory-based disease or disorder is hepatitis B virus infection. In another embodiment is the method wherein administering the composition is through oral, parenteral, intravenous, subcutaneous, intrathecal, intramuscular, buccal, intranasal, epidural, sublingual, pulmonary, local, rectal, or transdermal administration. In another embodiment is the method further comprising additional therapies selected from one or more of interferon α, pegylated interferon α-2a, lamivudine, adefovir, tenofovir, telbivudine and entecavir. In another embodiment is the method further comprising additional therapies selected from one or more Amikin, Avelox, Capastat, Cipro, levaquin, kantrex, Myambutol. In another embodiment is the method further comprising additional therapies selected from one or more of ActoPlus Met, Amaryl, Avandia, Byetta, GlucopHage, Glucotrol, Glucovance, Humalog, Janumet, Kombiglyze XR, Lantus, Levemir, Novolog, Onglyza, Prandin, Tradjenta, Victoza, Welchol.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: Structure for CaFolate chelate and CaPterin chelate.

FIG. 2: Dipterinyl calcium pentahydrate (DCP) chelate.

FIG. 3: activity and neopterin excretion both were suppressed by CaPterin.

### DETAILED DESCRIPTION OF THE INVENTION

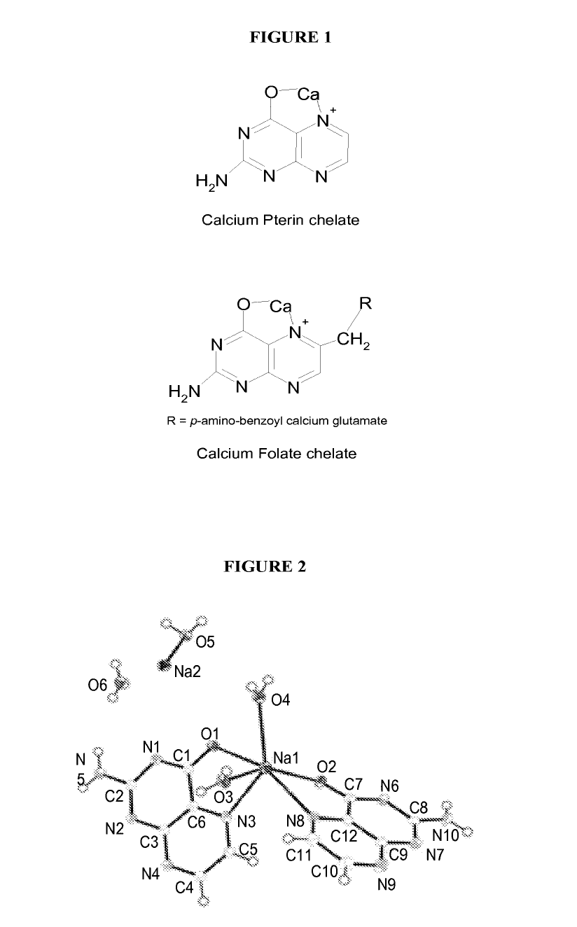

CaFolate and Folic acid works with vitamin B12 and vitamin C to help the body break down, use, and make new proteins. The vitamin helps form red blood cells. It also helps produce DNA, the building block of the human body, which carries genetic information. Folate, or folic acid, is a widely recognized vitamin that has been proven to cure a host of human ailments (National Center for Biotechnology Information USNLoM, Folate Deficiency, PubMed Health, 2011). Studies have in nude mice with MDA-MB-231 human breast xenograft tumors with CaFolate have shown anti-tumor activity. The tumor shrank in these mice possessing high levels of endogenous folate and consumed a level of dietary folic acid during the course of treatment. The structure of CaFolate has the same hetro-aromatic ring than CaPterin (FIG. 1). It has been observed that under acidic conditions, UV-irradiation of folic acid (pteroylglutamic acid) causes its successive oxidative cleavage to pterin-6-aldehyde, then to pterin-6-carboxylic acid, and finally to pterin as the end product (Lowry O H, Bessey O A and Crawford E J. PHotolytic and enzymatic transformations of pteroylglutamic acid. J Biol Chem. 1949: 180: 389-98).

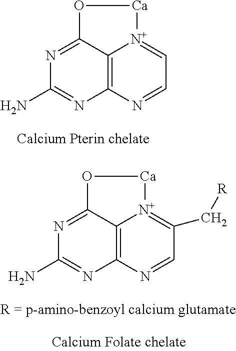

FIG. 1: Structure for Calcium Folate chelate and Calcium Pterin chelate

Previously it has been reported that Pterin is an immuno-modulator present in the blood and tissues of mammals. It is excreted in the urine of cancer patients in elevated amounts relative to normal persons (Sten B, Halpern R M, Halpern B C and Smith R A, Urinary excretion levels of unconjugated pterins in cancer patients and normal individuals. Clin Can Acta. 1981; 113: 231-42). When combined with calcium, oral Pterin demonstrates anti-tumorgenic (Moheno P, Pfleiderer W, DiPasquale A G, Rheingold A L and Fuchs D. Cytokine and IDO metabolite changes effected by calcium pterin during inhibition of MDA-MB-231 xenograph tumors in nude mice. Int J PHarm. 2008; 355: 238-48; Moheno P, Pfleiderer W and Fuchs D. Plasma cytokine concentration changes induced by the antitumor agents dipterinyl calcium pentahydrate (DCP) and related calcium pterins. Immunobiology. 2009; 214: 135-41; Moheno P B. Calcium pterin as an antitumor agent. Int J PHarm. 2004; 271: 293-300), anti-viral such as hepatitis B (Moheno P, Morrey J and Fuchs D. Effect of dipterinyl calcium pentahydrate on hepatitis B virus replication in transgenic mice. J Transl Med. 2010; 8: 32), anti-diabetic (Nikoulina S E, Fuchs D and Moheno P. Effect of Orally Administered Dipterinyl Calcium Pentahydrate (DCP) on Oral Glucose Tolerance in DIO Mice. (Diabetes, Metabolic Syndrome and Obesity: Targets and Therapy, 2012; in press) and anti-mycobacterial (BCG) activity in an in vitro model of tuberculosis (Sakala I G, Blazevic A, Moheno P and Hoft D F. Dipterinyl Calcium Pentahydrate inhibits intracellular mycobacterial growth in human monocytes via the C—C chemokine MIP-1beta and Nitric Oxide. Unpublished manuscript. 2011). It has been reported that a promising new cancer therapeutic, dipterinyl calcium pentahydrate (DCP), a dimer of pterin linked together with calcium (DCP) (FIG. 2), shows the same immune-modulatory activities as the monomer CaPterin such as anti-tumor, anti-infective and anti-diabetic activity.

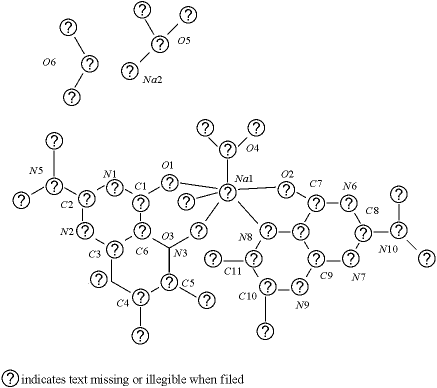

FIG. 2: X-ray crystallographic structure of dipterinyl calcium pentahydrate (DCP)

The sensitivity of CaFolate to acidic conditions, suggests that in-vivo, under acidic conditions it is transformed into CaPterin and exhibits the same profile of biological efficacies as CaPterin and DCP.

Materials and Methods

### EXAMPLE 1

In Vivo Tumor Studies

A series of in vivo studies provided initial evidence for an immunologically mediated antitumor mechanism for oral 1:4 mol:mol calcium pterin [CaPterin] in suspension (Moheno P B. Calcium pterin as an antitumor agent. Int J PHarm. 2004; 271: 293-300).

First, six- to eight-month-old C3H/HeN-MTV+ female mice, retired breeders, with a high propensity (˜90%) to develop mammary gland adenocarcinomas within a few weeks after their arrival, were received from the NCI. As each mouse developed a palpable tumor, it was assigned alternately to either Test or Control groups. The mice in the Test group received 3/16 ml of the CaPterin suspension (7 mg/kg/day) by oral gavage for seven days. The ratio of Test tumor volumes to Control tumor volumes (T/C) at Day 7 was 0.1 or 10%.

Second, athymic nude (nu/nu) female mice, age three to four weeks, were injected subcutaneously with 5×10⁶ MDA-MB-231 human breast cancer cells into the right leg. When the tumors reached a mean diameter of 3-5 mm, the mice were divided into two groups, of eight members each. The mice were treated by oral gavage once daily for 14 days with either 3/16 ml of the vehicle control (deionized H₂O) or with 3/16 ml of the CaPterin suspension (7 mg/kg/day). The mean V/Vo was plotted as a function of time after treatment, giving a T/C=0.41 or 41% after 14 days. No treatment toxicity was found as assessed by reductions in body weight during and after dosing.

Third, Balb/c female mice, age three to four weeks, were implanted subcutaneously in the right flank with 2×10⁷ EMT6 mouse mammary tumor cells. The mice were treated once daily for 15 consecutive days with either an oral injection of vehicle control ( 3/16 ml deionized H2O) or 7 mg/kg/day of 3/16 ml CaPterin suspension. The CaPterin suspension treatment produced no significant effect on tumor growth in the Balb/c mice with EMT6 allograpHs, and no measurable animal toxicity, as determined by decreased body weight.

Fourth, oral CaPterin suspension was tested in SCID mice bearing the human breast tumor cell line MDA-MB-231. Thirty-two SCID mice were inoculated with scraped MDA-MB-231 human breast cancer cells in matrigel using a subcutaneous flank injection. The mice were randomly assigned, eight mice to each of the following treatment groups: Control (distilled water); 13 mg/kg CaPterin; 20 mg/kg CaPterin; and 26 mg/kg CaPterin. Administration of either CaPterin or the vehicle by oral gavage was from Monday through Friday for 75 days. The CaPterin suspension showed no antitumor efficacy in the SCID mice. The experimental SCID mice demonstrated no measurable toxicity over the 75 days of CaPterin suspension administration.

Taken together, these results indicate CaPterin's antitumor activity is immunologically mediated, via indoleamine 2,3-dioxygenase (IDO) tumor escape mechanism (Uyttenhove C, Pilotte L, Theate I, et al. Evidence for a tumoral immune resistance mechanism based on tryptophan degradation by indoleamine 2,3-dioxygenase. Nat Med. 2003; 9: 1269-74).

### EXAMPLE 2

In Vitro Studies

Test for the IDO inhibition (Winkler C, Schroecksnadel K, Moheno P, Meebergen E, Schennach H and Fuchs D. Calcium-pterin suppresses mitogen-induced tryptophan degradation and neopterin production in peripheral blood mononuclear cells. Immunobiology, 2006; 211: 779-84).

Effect of CaPterin on freshly isolated human peripheral blood mononuclear cells (PBMC) stimulated with the mitogens phytohaemagglutinin and concanavalin A in vitro measure IDO (indoleamine 2,3-dioxygenase) activity, the kynurenine to tryptophan ratio (kyn/trp) was calculated and expressed as umol kynurenine/mmol tryptophan, both determined by High Pressure Liquid ChromatograpHy. Neopterin concentrations were determined by ELISA with a detection limit of 2 nmol/l.

Results

Table 1 shows in supernatants of unstimulated PBMC average concentrations of tryptophan and kynurenine were (mean±S.E.M.): 17.7±1.0 and 4.1±0.6 mmol/l, kyn/trp was 251±42.7 nmol/mmol). In the unstimulated PBMC, the addition of the CaPterin did not significantly affect concentrations of tryptophan nor of kynurenine, kyn/trp was lower in cells treated with the highest 200 mg/ml concentration (p<0.05). Concentration of neopterin was 8.1±0.7 nmol/l in unstimulated cells; the addition of the CaPterin did not influence this level.

Stimulation of cells with PHA decreased tryptophan concentrations to 0.2±0.1 mmol/l, in parallel, kynurenine concentrations increased to 14.4±1.4 mmol/l. Activation of IDO, as quantified by a decrease of tryptophan and a parallel increase of kynurenine concentrations and expressed as kyn/trp, was increased nearly 500-fold in stimulated compared with unstimulated PBMC (p<0.01). Stimulation of cells with Con A decreased tryptophan concentration to 0.4±0.1 mmol/l and increased kynurenine concentration to 14.4±1.6 mmol/l, resulting in significantly higher kyn/trp (p<0.01). The suspension of CaPterin suppressed stimulation-induced tryptophan degradation in a dose-dependent manner: tryptophan levels increased to baseline and kynurenine as well as kyn/trp significantly declined (≦100-fold decrease with PHA stimulation, p<0.001; and ≦500-fold decrease with Con A stimulation, p<0.001).

Stimulation of PBMC increased neopterin concentrations to 26.6±1.8 nmol/l for PHA and 43.7±7.5 nmol/l for Con A (both p<0.01). Addition of CaPterin to PHA- and Con A-stimulated cells had a significant suppressive effect (≦37% with PHA stimulation, p<0.001; and ≦58% with Con A stimulation, p<0.001).

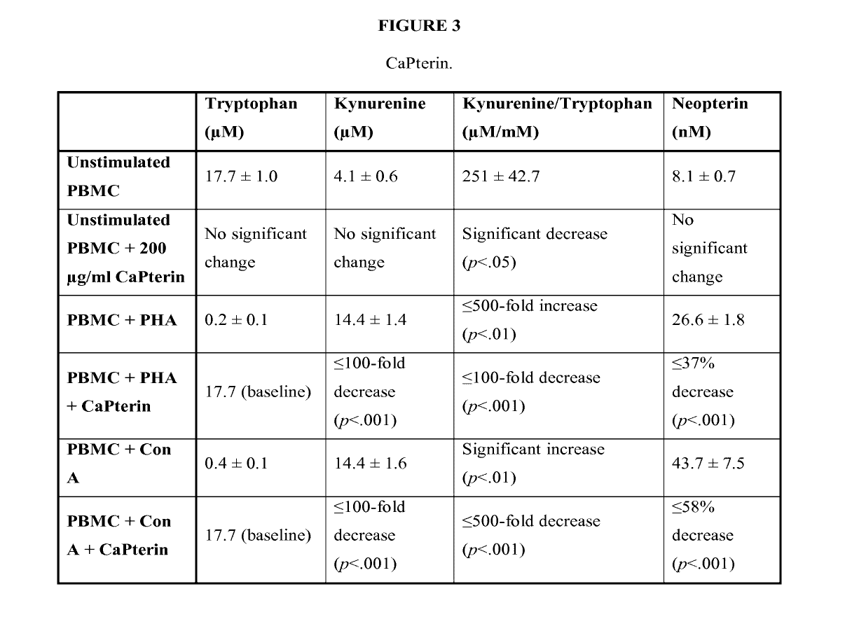

At the concentrations tested, no toxicity could be observed by the tryptophan blue exclusion method. FIG. 3: Table presenting IDO activity and neopterin excretion both were suppressed by CaPterin.

| | Tryptophan | Kynurenine | Kynurenine/Tryptophan | Neopterin |
|---|---|---|---|---|
| | (μM) | (μM) | (μM/mM) | (nM) |
| Unstimulated | 17.7 ± 1.0 | 4.1 ± 0.6 | 251 ± 42.7 | 8.1 ± 0.7 |
| PBMC | | | | |
| Unstimulated | No significant | No significant | Significant decrease | No significant |
| PBMC + 200 | change | change | (p < .05) | change |
| μg/ml CaPterin | | | | |
| PBMC + PHA | 0.2 ± 0.1 | 14.4 ± 1.4 | ≦500-fold increase | 26.6 ± 1.8 |
| | | | (p < .01) | |
| PBMC + PHA + | 17.7 (baseline) | ≦100-fold decrease | ≦100-fold decrease | ≦37% decrease |
| CaPterin | | (p < .001) | (p < .001) | (p < .001) |
| PBMC + Con | 0.4 ± 0.1 | 14.4 ± 1.6 | Significant increase | 43.7 ± 7.5 |
| A | | | (p < .01) | |
| PBMC + Con | 17.7 (baseline) | ≦100-fold decrease | ≦500-fold decrease | ≦58% decrease |
| A + CaPterin | | (p < .001) | (p < .001) | (p < .001) |

### EXAMPLE 3

In vivo MDA-MB-231 Tumor Studies (Moheno P, Pfleiderer W, DiPasquale A G, Rheingold A L and Fuchs D. Cytokine and IDO metabolite changes effected by calcium pterin during inhibition of MDA-MB-231 xenograph tumors in nude mice. Int J PHarm. 2008; 355: 238-48; Moheno P, Pfleiderer W and Fuchs D. Plasma cytokine concentration changes induced by the antitumor agents dipterinyl calcium pentahydrate (DCP) and related calcium pterins. Immunobiology. 2009; 214: 135-41)

Studies into the effectiveness of various forms of calcium pterin were next carried out in an experiment with the following aims,
1. To determine a dose response curve for the (1:4 mol/mol) calcium pterin [CaPterin] suspension.
2. To compare the antitumor activity of this suspension to pterin alone (pterin control).
3. To test the effect of CaPterin mega-dosing at 100 mg/(kg day).

Twenty-three athymic nude (nu/nu) female mice, ages 3-4 weeks, were injected subcutaneously with 5×10⁶ MDA-MB-231 cancer cells into the right flank. The four treatment groups were: 1:4 mol/mol) calcium pterin [CaPterin] (7 mg/(kg day)); pterin (21 mg/(kg day)); 1:4, mol/mol) calcium pterin [CaPterin] (21 mg/(kg day)); and sterile water control. CaPterin generated a dose-response relationship reaching a T/C=37% at 21 mg/(kg day) after 60 days of treatment. Pterin at 21 mg/(kg day) was found to have no antitumor activity.

DCP (dipterinyl calcium pentahydrate) was studied in another experiment with three aims:
1. To test the antitumor effect of the increased [Ca⁺²] in a (1:2 mol/mol) calcium pterin suspension as compared to the (1:4 mol/mol) calcium pterin [CaPterin] suspension;
2. To evaluate the antitumor efficacy of DCP at two concentrations, 23 and 69 mg/(kg day); and
3. To evaluate the antitumor activity of calcium chloride alone (CaCl₂ control).

In this experiment, 29 athymic nude were each injected subcutaneously with 10×10⁶ MDA-MB-231 cancer cells into the right flank. The five treatment groups were: (1:4 mol/mol) calcium pterin [CaPterin] (21 mg/(kg day)); (1:2 mol/mol) calcium pterin (25 mg/(kg day)); DCP (23 mg/(kg day)); DCP (69 mg/(kg day)); and calcium chloride dihydrate (4.2 mg/(kg day)). Blood was collected from all animals via cardiac puncture at termination (after 70-98 days of treatment) and processed to EDTA plasma for analysis. (1:2 mol/mol) calcium pterin (T/C=25%) and DCP at 23 and 69 mg/(kg d) (T/C=25% and T/C=50%; respectively) strongly inhibited MDA-MB-231 xenograph growth in the nude mice. Calcium chloride dihydrate also showed significant efficacy (T/C=25%) attributable to the high levels of endogenous folate-derived pterin in mice and to their dietary folate intake (HED=11 mg/day).

There was no observed toxicity, as determined by body weight changes, among any of the mice in either of these experiments. Moreover, there was no observed toxicity (appreciable Weight loss≧10%) among any of the mice mega-dosed by oral gavage with 100 mg/(kg day) CaPterin for up to 31 days.

A stepwise regression analysis of the following plasma measures: IL-1b, IL-2, IL-4, IL-6, IL10, IL-12, IFN-γ, TNF-α, kynurenine, tryptophan, and kyn/trp; yielded a CaPterin regression model significant to p=0.047:

CaPterin dose [mg/(kg d)]=10.5−0.096 [IL-6 pg/ml]+0.31 [IL-10 pg/ml]−3.16 [IFN-γ pg/ml]+7.89 [kyn μM]

This regression shows that CaPterin decreases IL-6 and IFN-y, and increases IL-10 and kynurenine. The kynurenine term is confounded by the fact that as tumors shrink, tryptophan plasma levels increase due to decreasing tumor cell growth demands, providing more IDO substrate.

A similar stepwise regression analysis for plasma IL-1b, IL-2, IL-4, IL-6, IL-10, IL-12, IFN-γ, and TNF-α; yielded a DCP antitumor plasma cytokine pattern (APCP) regression model significant to p=0.003:

DCP/APCP mg/(kg d))=7.235−0.002 [IL-12 pg/ml]−0.846 [IL-4 pg/ml]+0.051 [IL-6 pg/ml ]

This regression shows that for the DCP treated mice tumor growth, strongly correlated to DCP/APCP, decreases with IL-12 and IL-4, and increases with IL-6.

### EXAMPLE 4

In Vivo hepatitis B animal model study (Moheno P, Morrey J and Fuchs D. Effect of dipterinyl calcium pentahydrate on hepatitis B virus replication in transgenic mice. J Transl Med. 2010; 8: 32)

In this study with hepatitis B virus HMO transgenic mice, DCP was administered per os. once daily for 14 days at 23, 7.3, and 2.3 mg/(kg d). DCP caused a significant dose-response reduction of log liver HBV DNA as measured by PCR in the female HBV mice in the 2.3 to 23 mg/(kg d) range. 23 mg/(kg d)) DCP showed an 83% inhibition, comparable to adefovir dipivoxil (ADV) at 10 mg/(kg day). A stepwise regression of serum Tryptophan, Kynurenine, Kyn/Trp; HBV DNA [Southern], HBV DNA [PCR], HBV RNA [PCR], HBe antigen [ELISA]; Average # Liver HBcAg Nuclei per Total, Average # Liver HBcAg Cytoplasms per Total, Average # Liver HBcAg Nuclei per Quarter Field; IL-1a, IL-1b, IL-2, IL-3, IL-4, IL-6, IL-9, IL-10, IL-12, MCP-1, TNF-a, GM-CSF, RANTES, and liver IL-6; log HBV DNA [Southern], log rel. HBV DNA [PCR], log HBV RNA [PCR], and log HBe antigen [ELISA] gave the following DCP measured effects regression (p=0.001):

DCP dose (mg/(kg/d))=26.309−0.22 [MCP-1 rel. pg/ml]−4.065 [Log rel. HBV DNA (PCR)]−0.560 [kyn/trp uM/mM]+0.070 [GM-CSF rel. pg/ml]

Results: DCP decreased FIBV DNA as measured by PCR decreased the IDO activity serum kyn/trp. The chemokine MCP-1 was reduced.

Serum presence of GM-CSF was increased.

### EXAMPLE 5

In vitro study of DCP inhibition on intracellar mycobacterial growth in human monocytes (Sakala I G, Blazevic A, Moheno P and Hoft D F. Dipterinyl Calcium Pentahydrate inhibits intracellular mycobacterial growth in human monocytes via the C—C chemokine MIP-1beta and Nitric Oxide. Unpublished manuscript. 2011)

Tuberculosis remains one of the top three leading causes of morbidity and mortality.

Worldwide; complicated by the emergence of drug-resistant Mycobacterium tuberculosis strains and high rates of HIV co-infection. In this study, the ability of DCP to mediate killing of intracellular mycobacteria within human monocytes was tested. DCP treatment of infected monocytes resulted in a significant reduction in viability of intracellular but not extracellular M. bovis BCG. DCP potentiated monocyte antimycobacterial activity by induction of the C—C chemokine MIP-1β, and inducible nitric oxide synthase 2.
Addition of human anti-MIP-1β neutralizing antibody or a specific inhibitor of the L-arginase-nitric oxide pathway (L-NMMA monoacetate), reversed the inhibitory effects of DCP on intracellular mycobacterial growth.
Results
DCP induced mycobacterial killing via MIP-1β and nitric oxide dependent effects. Hence, DCP acts as an immunoregulatory compound enhancing the anti-mycobacterial activity of human monocytes. IDO gene expression was also suppressed in the infected monocytes by DCP.

### EXAMPLE 6

In Vivo study of orally administered DCP on Oral Glucose Tolerance in DIO Mice (Diabetes, Metabolic Syndrome and Obesity: Targets and Therapy. 2012; in press)

DCP as a novel therapeutic for Type 2 diabetes. Female DIO mice, C57BL/6J, fed a high-fat diet were administered DCP orally in 0.4% carboxymethylcellulose for 21 days. Blood glucose was followed during the dosing period, and an oral glucose tolerance test (OGTT) was carried out on day 21 after DCP administration, along with measurements of plasma indoleamine 2,3-dioxygenase (IDO) metabolites (tryptophan and kynurenine), and certain cytokines and chemokines (GM-CSF, IFNγ, IL-1α, IL-1β, IL-4, IL-6, IL-10, IL-12(p40), IL-12(p70), IL-13, MCP-1, RANTES, and TNFα). 7 mg/(kg d) DCP reduced OGTT/AUC (area under OGTT curve) by 50% (p<0.05). A significant multivariate regression p=0.013; R²=0.571) of OGTT/AUC was derived from DCP dosage and plasma tryptophan:

GTT/AUC=0.009 DCP³+31.178 DCP²−574.513 DCP+29.828 Trp+1935.382

Elevated plasma tryptophan was found to correlate with higher OGTT/AUC diabetic measures, possibly via inhibition of histamine degradation.

Conclusion:

An optimum dose of 7 mg/(kg d) DCP significantly improved the OGTT diabetic state in these female DIO mice.

We claim:

1. A method of treating cancer comprising administration of a composition comprising CaFolate.

2. The method of claim 1 wherein the CaFolate is CaPterin.

3. The method of claim 1 wherein the CaFolate is dipterinyl calcium pentahydrate (DCP).

4. The method of claim 1 wherein administration of the composition results in decreased IL-6 levels.

5. The method of claim 1 wherein administration of the composition results in increased IL-10 levels.

6. The method of claim 1 wherein administration of the composition results in decreased IFN-γ levels.

7. The method of claim 1 wherein administration of the composition results in increased kynurenine levels.

8. The method of claim 1 wherein administration of the composition results in increased IL-12 levels.

9. The method of claim 1 wherein administration of the composition results in decreased IL-6 levels.

10. The method of claim 1 wherein administration of the composition results in increased IL-4 levels.

11. The method of claim 1 wherein administration of the composition results in inhibition of indoleamine 2,3-dioxygenase.

12. The method of claim 1 wherein administering the composition is through oral, parenteral, intravenous, subcutaneous, intrathecal, intramuscular, buccal, intranasal, epidural, sublingual, pulmonary, local, rectal, or transdermal administration.

13. The method of claim 1 further comprising additional therapies selected from one or more of radiation therapy, chemotherapy, high dose chemotherapy with stem cell transplant, hormone therapy, and monoclonal antibody therapy.

14. The method of claim 1 wherein the cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

15. A method of modulating the immune response comprising administration of a composition comprising CaFolate.

16. A method of treating an inflammatory-based disease or disorder comprising administration of a composition comprising CaFolate.

17. The method of claim 16 wherein the inflammatory-based disease or disorder is selected from infectious diseases, neurodegenerative disorders, multiple sclerosis, HIV-associate dementia, AIDS dementia, Alzheimer's disease, central nervous system inflammation, obesity, dementia (various forms), coronary heart disease, diabetes (Type 1 and Type 2), atherosclerosis, chronic inflammatory diseases, autism, neonatal onset multisystem inflammatory disease, (also known as NOMID, Chronic Neurologic Cutaneous and Articular Syndrome, or CINCA), Parkinson's Disease, rheumatoid arthritis, osteoarthritis, tendinitis, bursitis, inflammatory lung disease, psoriasis, chronic obstructive pulmonary disease, lupus erythematosus, organ inflammation (e.g. myocarditis, asthma, nephritis, colitis), inflammatory bowel disease (IBD), autoimmune disease, inflammatory bowel syndrome (IBS), Crohn's Disease, Chronic Ulcerative Colitis, transplant rejection, sepsis, disseminated intravascular coagulation (DIC), septic shock, psoriasis, emphysema and ischemia-reperfusion injury.

18. The method of claim 16 wherein the inflammatory-based disease or disorder is hepatitis B virus infection.

19. The method of claim 16 wherein the inflammatory-based disease or disorder is mycobacterial infection.

20. The method of claim 19 wherein administration of the composition potentiate C—C Chemokine MIP-1β.

21. The method of claim 19 wherein administration of the composition induce nitric oxide synthase 2.

22. The method of claim 16 wherein the inflammatory-based disease or disorder is type-2 diabetes.

23. The method of claim 22 wherein administration of the composition results in decreased Oral Glucose Tolerance Test Area-under-curve (OGTT/AUC).

24. The method of claim 22 wherein administration of the composition results in decreased IL-6 levels.

25. The method of claim 22 wherein administration of the composition results in decreased MCP.

26. The method of claim 16 wherein administering the composition is through oral, parenteral, intravenous, subcutaneous, intrathecal, intramuscular, buccal, intranasal, epidural, sublingual, pulmonary, local, rectal, or transdermal administration.

27. The method of claim further comprising additional therapies selected from one or more of interferon α, pegylated interferon α-2a, lamivudine, adefovir, tenofovir, telbivudine and entecavir.

28. The method of claim 19 further comprising additional therapies selected from one or more of Amikin, Avelox, Capastat, Cipro, levaquin, Kantrex, Myambutol.

29. The method of claim 22 further comprising additional therapies selected from one or more of
ActoPlus met, Amaryl, Avandia, Byetta, Glucophage, Glucotrol, Glucovance, Humalog, Janumet, Kombiglyze XR, Lantus, Levemir, Novolog, Onglyza, Prandin, Tradjenta, Victoza, Welchol.

30. The method of claim 16 wherein the CaFolate is dipterinyl calcium pentahydrate (DCP).

31. The method of claim 16 wherein the CaFolate is CaPterin.
